(19) United States
(12) Patent Application Publication
(10) Pub. No.: US 2020/0037926 A1
(43) Pub. Date: Feb. 6, 2020

(54) Gait Training Method with a Sensor-Based Reduction or Variation of the Weight Load

(71) Applicant: BEKA Hospitec GmbH, Wetzlar, DE (US)

(72) Inventor: Matthias TOMCZAK, Frankfurt am Main (DE)

(21) Appl. No.: 16/492,054

(22) PCT Filed: Mar. 7, 2018
(86) PCT No.: PCT/EP2018/055674

§ 371 Date: Sep. 6, 2019

(30) Foreign Application Priority Data
Mar. 9, 2017 (DE) 10 2017 105 065.5
Mar. 9, 2017 (DE) 102017105065.5

(51) Int. Cl. A61B 5/11; A61B 5/00; A61H 3/00; A61B 5/0488

(52) U.S. Cl. CPC A61B 5/112; A61B 5/7282; A61H 3/008; A61B 5/0488; A61B 5/1071; A61H 2201/1652; A61H 2201/5007; A61H 2201/5061; A61B 2505/09

(57) ABSTRACT

A gait training method includes a sensor-based reduction or variation of the weight load (16) in order to improve the gait (21) of an individual (2). A device (1) is used to reduce or vary the weight load (16). The device (1) has a sensor system (3a-e) which detects physical and biometric characteristic data (12), in particular in real-time and automatically via different sensors, and the CPG activity via surrogate signals as additional characteristic data (13) of the individual (2). The characteristic data (12, 13) is used to regulate and/or control a reduction or variation of the weight load (16).

### CROSS-REFERENCE TO RELATED APPLICATIONS

This application is a filing under 35 U.S.C. 371 of International Application No. PCT/EP2018/055674 filed Mar. 7, 2018, entitled “Gait Training Method with a Sensor-Based Reduction or Variation of the Weight Load,” which claims priority to German Patent Application No. 10 2017 105 065.5 filed Mar. 9, 2017, which applications are incorporated by reference herein in their entirety.

### FIELD OF INVENTION

The present disclosure relates to a method for carrying out a gait training or a gait analysis for improving the gait of an individual.

### BACKGROUND

The prior art has proposed various methods for carrying out a gait training and/or a gait analysis for improving the gait of an individual, in which use is made of an apparatus that captures different biometric and/or physical characteristics of the individual and that is able, where necessary, to accordingly reduce or vary the weight loads on the individual.

Thus, WO 2014/153201 A1, for example, discloses a method that resorts to an apparatus that measures biometric and physical characteristics of an individual by various sensors for the purposes of improving the gait of the individual. Here, an air pressure-controlled holding system, into which the individual is placed, assists walking or running by actively lifting the body of the individual.

The holding system can be subject to closed-loop control in automatic fashion and depending on the measured characteristics, with the sensors capturing biometric and/or physical data such as walking speed, leg and foot position, ground reaction, stepping force, circulatory system and/or muscle activity, for example. A computer connected to the sensor system can derive the overall load on the individual from the measured data and can calculate the extent to which the holding system should lift the body of the individual and consequently ensure a reduction or variation in the weight load.

A substantial disadvantage of this method consists of the fact that the apparatus employed therein can measure individual gait phases, in particular the so-called stance phase, by way of video sensors only. Although the gait phases can be subsequently analyzed on the basis of these video recordings, the video recordings themselves do not influence the reduction or variation of the weight loads in any way. Closed-loop control is implemented purely on the basis of the measured overall load on the individual and not on account of specific pulses or aspects from the individual gait phases; i.e., the known solution for a weight-unburdened gait training consequently always unburdens the body in constantly equal fashion. No adaptation of or reduction in the weight load to individual gait phases of the individual, such as the stance phase, for example, is possible.

However, all of this does not usually correspond to the real situation of “normal” walking and may lead to gait therapies that resort to such a method, for instance, yielding unsatisfactory results.

### SUMMARY OF INVENTION

The present disclosure provides an improved method for carrying out a gait training or a gait analysis for the purposes of improving the gait of an individual or patient. Moreover, in addition to the targeted measurement of characteristics, the method also allows the individual gait phases to be influenced, in particular depending on the characteristics respectively ascertained in the individual gait phases.

In a method for carrying out a gait training and/or a gait analysis for improving or modifying the gait of an individual using an apparatus for reducing or varying weight loads on the individual, wherein the method comprises the capture of physical and/or biometric characteristics of the individual, wherein the characteristics are captured by a physical and/or physiological sensor system, and wherein there is a closed-loop and/or open-loop control of the reduction or variation of weight loads depending on the captured characteristics, the CPG activity in the spinal cord of the individual can be captured by measuring surrogate signals that are used as additional characteristics for closed-loop and/or open-loop control of the apparatus for reducing or varying the weight loads.

The CPG activity controls the rhythmization of the gait pattern together with higher motor centers (motor neuron networks in the brain). Capturing the CPG activity by way of surrogate signals facilitates the measurement or the capture of individual gait phases of the individual in a surprisingly simple manner. Depending on this measurement or capture there is a targeted and dynamic adaptation in the reduction or variation of the weight load during the individual gait phases, as a result of which the CPG activity can be influenced in a targeted fashion in turn, leading to an improvement in the gait of the individual. In connection therewith, the method according to the disclosure advantageously facilitates taking account of the natural load peaks of the stance phase (heel strike and toe off) that serve as initial firing of the rhythmizing networks (spinal cord and motor neuron networks in the brain) when varying the weight load during the gait phase. This is reflected in an activation and a better rhythmization of these neural networks. This improved rhythmization can also be observed straightaway by way of a subsequent renewed measurement or capture of the surrogate signals, from which immediate feedback arises about the therapy or training success.

The automated closed-loop and/or open-loop control of the apparatus for reducing or varying the weight load by way of the additional characteristics further leads to immediate feedback being obtained about the ability of the individual relating to the amount of weight they can just still support on a standing leg. This information is transferred to the closed-loop and/or open-loop control of the weight unburdening, leading to a significant improvement in the gait training or in the gait analysis.

It is possible to identify that the basic concept of the method for carrying out a gait training and/or a gait analysis for improving or modifying the gait of an individual consists in capturing characteristics of the individual by way of a physical and/or physiological sensor system and obtaining additional characteristics of the individual from the CPG activity and introducing these characteristics directly into an apparatus for reducing or varying weight loads on the individual in such a way that the measured characteristics and/or the additional characteristics directly influence the closed-loop and/or open-loop control of the reduction or variation in the weight load by the apparatus. This implements not only an accurate measurement and capture of a gait phase, specifically the stance phase. There is a dynamic adaptation of the reduction of the variation of the weight load during the gait phase, more particularly the stance phase, specifically during individual load peaks within the stance phase (heel strike and toe off) from the closed-loop and/or open-loop control of the reduction or variation of weight loads depending on the captured characteristics in combination with the capture of additional characteristics. The result is an immediately perceptible and recognizable improvement in the gait of the individual.

In a preferred variant, provision is made for the characteristics and the additional characteristics to be captured in real time. As a result of this real-time capture a virtually real map of the gait situation of an individual can be produced on the basis of individual or combined characteristics, said map of the gait situation being used to control the apparatus for reducing or varying the weight load in dynamic fashion and in targeted fashion during individual gait phases.

The characteristics of the individual may comprise their body weight, a walking speed, a ground reaction, a stepping force, a leg and/or foot position, a joint angle, measurement values of the cardiovascular system, a muscle activity or the like. Thus, these may be biomechanical, kinetic, kinematic, neural or biometric characteristics. Here, provision is made, in particular, for gait phases to be reliably detected by various sensors and being linked to an electromyographic (EMG) activity.

Gait situation and physical load on the individual should be mapped as comprehensively as possible thereby. As a result, there can be a targeted and dynamic adaptation in the reduction or variation of the weight load during the individual gait phases by virtue of the apparatus for reducing or varying the weight load being actuated in a targeted fashion and in real time.

An advantage of the disclosure is that the level of fatigue of the individual can be determined by way of the characteristics and/or the additional characteristics. Specifically, voluntary joint actions of the individual can be correlated with measured movement data. This ensures a dynamic adaptation of the reduction or variation in the weight load.

Further, provision is made for the surrogate signals to comprise electromyographic signals of antagonists and/or agonists involved in the gait. The method consequently facilitates the capture of the activity of the central pattern generators by way of electromyographic surrogate signals. Measuring the surrogate signals consequently represents an easy-to-apply option for capturing the CPG activity.

A further variant of the method provides for the closed-loop and/or open-loop control of the reduction or variation of weight loads depending on the captured characteristics and/or the additional characteristics to be implemented in automatic and/or dynamic fashion.

Consequently, the human-machine-system arising thereby no longer requires external control by further persons, such as a therapist, for example, for the training or therapy sessions. Moreover, the closed-loop and/or open-loop control depending on the captured characteristics, which is implemented in real time and automatically, ensures that there is a dynamic reduction or variation in the weight load. By way of the dynamic reduction or variation of the weight load, it is possible to address at least three gait phases, such as, e.g., the “heel strike”, “mid-phase” and/or “toe off” gait phases, within the stance phase of a gait cycle in targeted fashion; this is not possible using the methods specified in the prior art. The transferability to real walking, which is improved in comparison with conventional gait training apparatuses, leads the individual to a stable and economical gait pattern within a shorter period of time.

Provision is further made for the closed-loop and/or open-loop control of the reduction or variation of weight loads to be implemented by a closed-loop and/or open-loop control unit. The dynamic adaptation of the reduction or variation in the weight load can be imparted by the closed-loop and/or open-loop control unit.

Moreover, there can be a targeted reduction or variation in the weight loads on the individual depending on the additional characteristics, obtained by way of the surrogate signals, in the case of load peaks in a stance phase. This is advantageous in that spinal and/or supraspinal neural networks of the individual are initiated. By way of example, spinal networks are the central pattern generators (CPG) in the spinal cord; by way of example, supraspinal neural networks are the motor centers in the brain. These networks control the rhythmization of the gait pattern. Targeted adaptation peaks of the weight loads during the stance phase consequently bring about an optimization of the gait pattern and ensure better training or therapy successes.

In particular, an improved gait pattern of the individual can be produced from the captured characteristics and/or the additional characteristics. There can be a dynamic adaptation of the reduction or variation of the weight loads on the basis of the improved gait pattern. The consequence of this is an improvement in the gait of the individual.

According to the method according to the disclosure, the sensor system can comprise kinetic, kinematic, electromyographic, thermal, optical and/or tactile sensors. This allows the improved mapping of the gait pattern of the individual to be ensured.

Specifically, the method can provide for the sensor system to comprise inertial sensors, pressure sensors, light sensors, weight sensors, force sensors, array sensors, needle electrodes, surface electrodes, video cameras or the like.

In some embodiments, the characteristics and/or additional characteristics captured by way of the sensor system can be transferred to a computer. A consequence of this is that the apparatus for reducing or varying weight loads on the individual can be used without a further person, such as a therapist, for example.

The method according to the disclosure can provide for the characteristics and/or the additional characteristics to be pre-processed in the computer by a processor. Here, the characteristics can be filtered and/or compressed in context-based fashion dependent on current requirements. As a result, a better and faster dynamic adaptation of the reduction or variation of the weight loads is facilitated.

The transfer of the characteristics and/or the additional characteristics from the sensor system to the computer can be implemented in wireless fashion in an advantageous variant. As a result of dispensing with transfer cables, the apparatus for reducing or varying the weight loads can be connected and operated more easily. The consequence of this is more user-friendly handling.

The method can provide for the computer to monitor a closed-loop and/or open-loop control unit. This can be implemented automatically and in real time. This likewise is advantageous in that the apparatus for reducing or varying weight loads on the individual can be used without a further person.

In particular, the method can provide for monitoring of the closed-loop and/or open-loop control unit by the computer to be implemented in wireless fashion. As a result of this, the apparatus for reducing or varying the weight loads can be connected and operated more easily. The consequence of this is more user-friendly handling.

In particular, the method can provide for the individual to be held in a holder of the apparatus by a belt system, wherein the belt system is able to carry the individual, for example in the region of the hips and/or in the region of the torso. Here, the belt system and the holder can be movable in at least one direction. The belt system and the holder can ensure that the individual can move freely to a large extent and at the same time experience assistance with the gait. This assistance may comprise a stabilization and/or a reduction or variation in the weight load on the individual and it is advantageous for the improvement of the gait.

### BRIEF DESCRIPTION OF THE DRAWINGS

Further features, details and advantages of the invention emerge from the phrasing of the claims and from the following description of exemplary embodiments on the basis of the drawings. In detail:

FIG. 1 shows a method chart with a schematic representation of a method for carrying out a gait training or a gait analysis for improving the gait of an individual, and

FIG. 2 shows a schematic illustration of an apparatus for carrying out the method for carrying out a gait training or gait analysis for improving the gait of an individual.

### DETAILED DESCRIPTION OF THE DRAWINGS

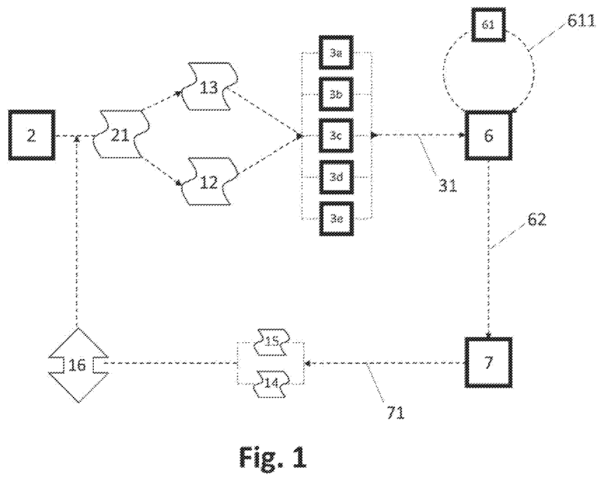

FIG. 1 shows a method chart for carrying out a gait training or a gait analysis for improving the gait 21 of an individual 2 in exemplary fashion.

Initially, the individual 2 starts by carrying out a gait 21. In the process, the characteristics 12 and/or the additional characteristics 13 of the individual 2 are captured by the sensor system 3a-e, wherein the sensor system 3a-e comprises kinematic, kinetic and electrophysiological sensors that can form a sensor network together.

The characteristics 12 and/or additional characteristics 13, which are captured by the sensor network, are initially transferred to a computer 6 for closed-loop and/or open-loop control 71 of the reduction or variation of the weight load 16. This transfer 31 can be implemented automatically, in real time and in wireless fashion. Advantageously, the computer 6 comprises a processor 61 that is able to pre-process the transferred characteristics 12, 13. By way of example, pre-processing 611 may include filtering the characteristics 12, 13 or filtering out background signals or the like. Moreover, the characteristics 12, 13 can be compressed.

In the example of FIG. 1, the pre-processed characteristics 12, 13 are used to control or regulate a reduction or variation of the weight load 16, wherein the computer 6 monitors the closed-loop and/or open-loop control unit 7 depending on the pre-processed characteristics 12, 13. Monitoring 62 of the closed-loop and/or open-loop control unit 7 can be implemented automatically, in real time and/or in wireless fashion. In a preferred variant, the latency time that elapses between capturing the characteristics 12, 13 and closed-loop and/or open-loop control 71 of the variation or reduction of the weight load 16 is no more than 50 milliseconds.

Furthermore, the closed-loop and/or open-loop control 71 can be implemented dynamically depending on the characteristics 12, 13. The dynamic closed-loop and/or open-loop control 71 makes it possible to assist and activate at least three gait phases such as, for example, the “heel strike”, “mid-phase” and/or “toe off” gait phases within the stance phase of a gait cycle in targeted fashion, as a result of which spinal and/or supraspinal neural networks of the individual are initiated.

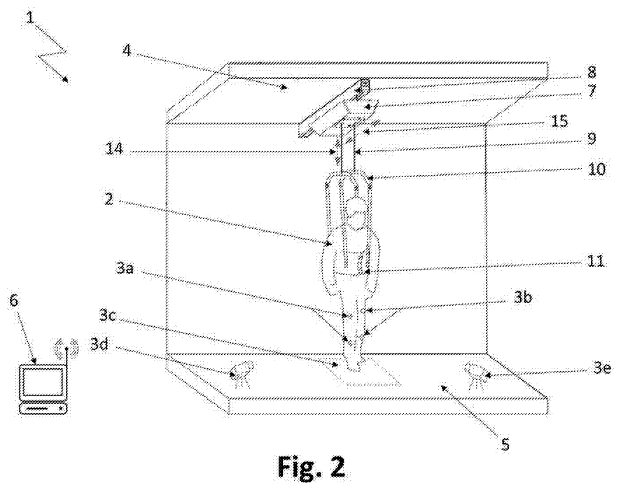

The closed-loop and/or open-loop control unit 7 regulates or controls the dynamic reduction or variation of the weight load 16 individual 2 in this example. Here, the closed-loop and/or open-loop control unit 7 ensures the dynamic reduction or variation of the weight load 16 on the individual 2 by way of a vertical movement direction 14 and/or a horizontal movement direction 15 using a holder 10 and a belt system 11 (see also FIG. 2 in this respect). This dynamic reduction or variation of the weight load 16 can be implemented in targeted fashion during individual load peaks in the stance phase, as a result of which spinal and/or supraspinal neural networks of the individual 2 can be initiated. This leads to an improvement in the gait of the individual 2.

In schematic fashion, FIG. 2 shows a gait training and/or a gait analysis being performed for the purposes of improving the gait 21 of an individual 2 using an apparatus 1 for reducing or varying the weight load 16 on the individual 2. In this example, the apparatus 1 comprises a ceiling element 4 and a floor element 5. The sensor system 3a-e of the apparatus 1 captures physical and biometric characteristics 12 by various sensors and the CPG activity as additional characteristics 13 of the individual 2 by way of surrogate signals.

The characteristics 12 and the additional characteristics 13 are transferred to the computer 6 in real time, automatically and/or in wireless fashion. A processor 61 that pre-processes the transmitted characteristics 12, 13 may be situated in the computer 6. Here, the pre-processing 611 comprises, e.g., filtering the characteristics 12, 13 or filtering out background signals or the like. Moreover, the pre-processing 611 may comprise a compression of the characteristics 12, 13.

The pre-processed characteristics 12, 13 are used to regulate a reduction or variation of the weight load 16, with the computer 6 monitoring the closed-loop and/or open-loop control unit 7 depending on the pre-processed characteristics 12, 13. In the shown example, the computer 6 is directly connected to the closed-loop and/or open-loop control unit 7. However, as illustrated in FIG. 2, the closed-loop and/or open-loop control unit 7 can also be monitored 62 in wireless fashion. In particular, provision is made here for the monitoring 62 to be implemented in real time and/or automatically. Furthermore, the closed-loop and/or open-loop control 71 can be implemented dynamically depending on the characteristics 12, 13.

The closed-loop and/or open-loop control unit 7 regulates or controls the reduction or variation of the weight load 16. In this example, the closed-loop and/or open-loop control 71 is implemented by a suspension 9, which is fastened to a rail system 8. In FIG. 2, the suspension 9 is connected to a holder 10. In turn, a belt system 11 that can hold the individual 2 is connected to the holder 10.

The rail system 8 is fastened to a ceiling element 4 in the illustrated example. The rail system 8 provides the closed-loop and/or open-loop control unit 7 with mobility in a horizontal movement direction 15. The variation or reduction in the weight load is implemented by way of a height adjustment of the suspension 9 by the closed-loop and/or open-loop control unit 7. Consequently, the suspension 9 is adjusted in the vertical movement direction 14, as a result of which the individual 2, who is connected to the suspension 9 by the belt system 11 and the holder 10, can be raised or lowered. By way of this dynamic closed-loop and/or open-loop control 71, it is possible to activate at least three gait phases, such as, e.g., the “heel strike”, “mid-phase” and/or “toe off” gait phases, within the stance phase of a gait cycle in targeted fashion depending on the captured characteristics 12 and the additional characteristics 13, as a result of which spinal and/or supraspinal neural networks of the individual 2 are initiated.

For the purposes of carrying out the method, the rail system 8 can have a length that is sufficient to implement a few steps of the gait of an individual 2 along the longitudinal axis of the rail system 8. The individual can carry out their gait on a treadmill or the like. Furthermore, a treadmill can be situated in the floor element 5.

The invention is not restricted to any of the embodiments described above but can be developed in multifaceted ways. Thus, the apparatus 1 need not have either a ceiling element 4 or a floor element 5. Instead, it can be installed directly in a room.

Furthermore, provision is not necessarily made for the holder 10 to be connected to a rail system 8 by way of a suspension 9. In an alternative embodiment, the holder 10 with the belt system 11 can be replaced by a support or holding system that grips or holds the individual 2 from the side or from below, as a result of which the rail system 8 and the suspension 9 can be dispensed with.

In a further embodiment, the rail system 8 can have a significantly longer embodiment in order to be able to produce longer gait movements or gait patterns. In a complementary or alternative fashion, the entire apparatus may also be arranged over a treadmill (not shown in any more detail) such that the individual 2 can carry out several or longer gait movements.

Further, the apparatus 1 can be operated automatically or by the individual 2 themselves or by a further person, such as a therapist, for example.

The gait training method comprises a sensor-based reduction or variation of the weight load 16 for improving the gait 21 of an individual 2, with use being made of an apparatus 1 for reducing or varying the weight load 16. The apparatus 1 comprises a sensor system 3a-e which captures physical and biometric characteristics 12 and the CPG activity via surrogate signals as additional characteristics 13 of the individual 2 by way of various sensors, more particularly in real time and automatically. The characteristics 12, 13 are used to control and/or regulate a reduction or variation of the weight load 16. Here, the closed-loop and/or open-loop control 71 can be implemented dynamically depending on the characteristics 12, 13. By way of the closed-loop and/or open-loop control 71 of the reduction or variation of the weight load 16, it is possible to activate at least three gait phases, such as, e.g., the “heel strike”, “mid-phase” and/or “toe off” gait phase, within the stance phase of a gait cycle in a targeted fashion, as a result of which spinal and/or supraspinal neural networks of the individual 2 are initiated. The closed-loop and/or open-loop control of the reduction or variation of the weight load 16 is implemented by a holder 10, a belt system 11 that can hold the individual being connected to the holder 10.

All features and advantages emerging from the claims, the description and the drawing, including structural details, spatial arrangements and method steps, can be essential to the invention, both on their own and in the various combinations.

### LIST OF REFERENCE DESIGNATIONS

1 Apparatus
2 Individual
3a-e Sensor system
4 Ceiling element
5 Floor element
6 Computer
7 Closed-loop and/or open-loop control unit
8 Rail system
9 Suspension
10 Holder
11 Belt system
12 Characteristics
13 Additional characteristics
14 Vertical movement direction
15 Horizontal movement direction
16 Reduction or variation of the weight load
21 Gait
31 Transfer
61 Processor
62 Monitoring
71 Closed-loop and/or open-loop control
611 Pre-processing

1. A method for carrying out a gait training and/or a gait analysis for improving or modifying the gait of an individual
using an apparatus for reducing or varying weight loads on the individual,
wherein the method comprises the capture of physical and/or biometric characteristics of the individual,
wherein the characteristics are captured by a physical and/or physiological sensor system, and
wherein there is a closed-loop and/or open-loop control of the reduction or variation of weight loads depending on the captured characteristics,
characterized
in that the CPG activity in the spinal cord of the individual is captured by measuring surrogate signals that are used as additional characteristics for the closed-loop and/or open-loop control of the apparatus for reducing or varying the weight loads.

2. The method as claimed in claim 1, characterized in that the characteristics and/or the additional characteristics are captured in real time.

3. The method as claimed in claim 1, characterized in that the characteristics of the individual comprise their body weight, a walking speed, a ground reaction, a stepping three, a leg and/or foot position, a joint angle, measurement values of the cardiovascular system, a muscle activity or the like.

4. The method as claimed in claim 1, characterized in that the level of fatigue of the individual is determined by way of the characteristics and/or the additional characteristics.

5. The method as claimed in claim 1, characterized in that the surrogate signals comprise electromyographic signals of antagonists and/or agonists involved in the gait.

6. The method as claimed in claim 1, characterized in that the closed-loop and/or open-loop control of the reduction or variation of weight loads depending on the captured characteristics and/or the additional characteristics is implemented in automatic and/or dynamic fashion.

7. The method as claimed in claim 1, characterized in that the closed-loop and/or open-loop control of the reduction or variation of weight loads is implemented by a closed-loop and/or open-loop control unit.

8. The method as claimed in claim 1, characterized in that there is a targeted reduction or variation in the weight loads on the individual depending on the additional characteristics, obtained by way of the surrogate signals, in the case of load peaks in a stance phase.

9. The method as claimed in claim 1, characterized in that an improved gait pattern is produced from the captured characteristics and/or the additional characteristics.

10. The method as claimed in claim 1, characterized in that the sensor system comprises kinetic, kinematic, electromyographic, thermal, optical and/or tactile sensors.

11. The method as claimed in claim 1, characterized in that the sensor system comprises inertial sensors, pressure sensors, light sensors, weight sensors, force sensors, array sensors, needle electrodes, surface electrodes, or video cameras.

12. The method as claimed in claim 1, characterized in that the characteristics and/or additional characteristics captured by way of the sensor system are transferred to a computer.

13. The method as claimed in claim 12, characterized in that the characteristics and/or the additional characteristics are pre-processed in the computer by a processor.

14. The method as claimed in claim 12, characterized in that a transfer of the characteristics from the sensor system to the computer is implemented in wireless fashion.

15. The method as claimed in claim 7, characterized in that monitoring of the closed-loop and/or open-loop control unit is implemented in wireless fashion.
